(19) United States
(12) Patent Application Publication
(10) Pub. No.: US 2006/0258888 A1
(43) Pub. Date: Nov. 16, 2006

(54) Bis-Amination of Aryl Halides

(75) Inventors: Rogelio P. Frutos, Sandy Hook, CT (US); Isabelle Gallou, Paris (FR); Dhileepkumar Krishnamurthy, Brookfield, CT (US); Xiufeng Sun, Monroe, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim International, GmbH, Ingelheim (DE)

(21) Appl. No.: 11/380,956

(22) Filed: May 1, 2006

Related U.S. Application Data
(60) Provisional application No. 60/680,404, filed on May 12, 2005.

(51) Int. Cl. C07C 209/10

(52) U.S. Cl. 564/407

(57) ABSTRACT

Disclosed are methods for making 1,3- and 1,4-diamino-phenyl intermediates by utilizing bis-amination of ortho-substituted aryl halides.

### APPLICATION DATA

This application claims benefit to US Provisional application Ser. No. 60/680,404 filed May 12, 2005.

### BACKGROUND OF THE INVENTION

1. Technical Field

This invention relates to a process of making 1,3- and 1,4-diamino-phenyl intermediates using a bis-amination reaction.

2. Background Information

Aryl- and heteroaryl-substituted ureas have been described as inhibitors of cytokine production and effective therapeutics in cytokine-mediated diseases including inflammatory and autoimmune diseases. Examples of such compounds are reported in U.S. Pat. Nos. 6,080,763 and 6,319,921, and WO 00/55139 including aryl- or heteroaryl-substituted ureas

US publication number US 2004-102492 discloses heteroaryl amide compounds which are disclosed therein as being useful as cytokine inhibitors. Particular compounds disclosed in the publication are synthesized from arylamine intermediate compounds, such as N-[3-Amino-2-methoxy-5-(1-methyl-cyclopropyl)-phenyl]-methanesulfonamide. These arylamine intermediates are produced in a multistep process which require the synthesis of 1,3-diamino-phenyl intermediates, as shown in the scheme I below:

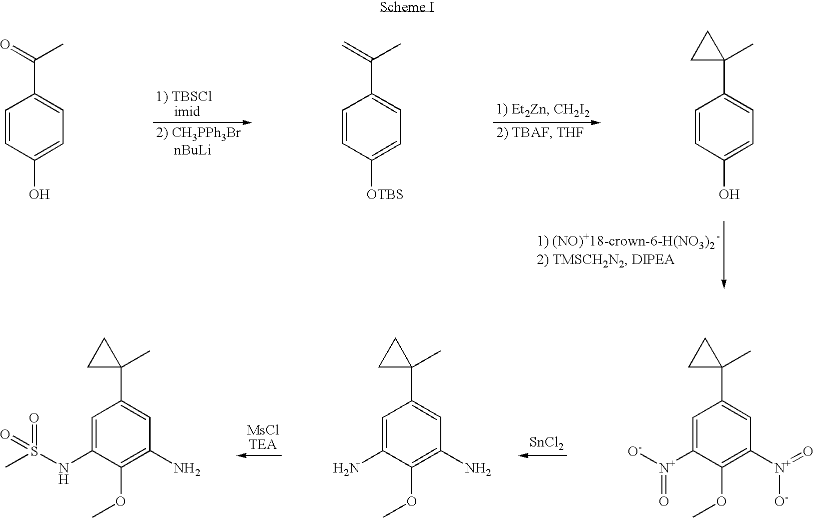

As seen in scheme I, the existing process uses functionalized di-nitrobenzene intermediates that decompose at relatively low temperatures and requires the use of expensive crown ether reagents. Similar reactions for these intermediates are disclosed in US 2004-0186114.

The amination of aryl halides has been disclosed in Lee S, et al., Org. Lett. 2001 3, 2729; Huang et al. Org. Lett. 2001, 3, 3417; and in Hartwig et al. WO 03/006420. However, lacking in the field are methods for bis-amination of ortho-substituted aryl halides.

It is therefore desirable to provide a more efficient and economical synthesis for 1,3-diamino-phenyl intermediates by utilizing bis-amination of ortho-substituted aryl halides.

### BRIEF SUMMARY OF THE INVENTION

It is therefore an object of the invention to provide a process of making 1,3-diamino-phenyl intermediates of the formula (I) via bis-amination of ortho-substituted aryl halides, 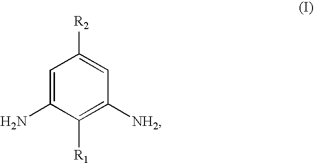
where R₁, R₂ and the suitable conditions of such process are described herein below.

### DETAILED DESCRIPTION OF THE INVENTION

In the broadest generic embodiment, there is provided a process of making 1,3- and 1,4-diamino-phenyl intermediates of the formulas (I) or (III) via bis-amination: 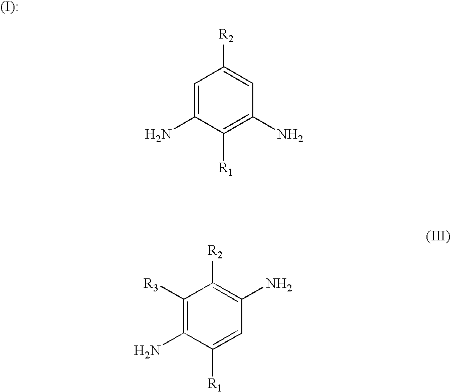
preferably, formula (I);
wherein
R₁ is chosen from hydrogen, C1-6 alkyl, aryl or C3-7 cycloalkyl each optionally substituted by C1-6 alkyl, C1-4 acyl, aroyl, C1-4 alkoxy, C1-6 alkoxycarbonyl each of the above may be partially or fully halogenated, carbocyclesulfonyl and —SO₂—CF₃;
R₂ is chosen from hydrogen, C1-6 alkyl, C3-7 cycloalkyl optionally substituted by C1-6 alkyl, C1-4 acyl, aroyl, C1-4 alkoxy, C1-6 alkoxycarbonyl each of the above may be partially or fully halogenated, carbocyclesulfonyl, halogen and —SO₂—CF₃;
wherein for formula II, R₃ and R₂ optionally fuse to form a benzo ring;
the process comprising in a one pot reaction:
providing an aryl halide of the formula (II) or (IV): 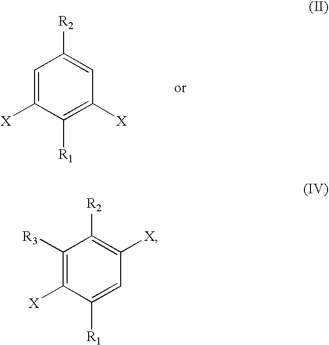
   wherein R₁, R₂, R₃ are as defined above, each X is independently halogen chosen from I and Br;
adding, in a suitable aprotic solvent including but not limited to toluene, THF, dioxane, preferably toluene;
an ammonia containing compound including but not limited to triphenylsilylamine tri-n-hexylsilylamine, trimethylsilylamine, t-butyl carbamate, benzyl carbamate, preferably triphenylsilylamine;
a palladium containing compound including but not limited to Pd₂(dba)₃, Pd(dba)₂, Pd(OAc)₂ PdCl₂, [(allyl)PdCl]₂, preferably Pd₂(dba)₃;
a phosphine containing compound including but not limited to 2-(dicyclohexylphosphino)biphenyl, triphenylphosphine, tri-t-butylphosphine, BINAP, DPPF, preferably 2-(dicyclohexylphosphino)biphenyl;
and LiHMDS (lithium bis-trimethylsiloamide);
at a temperature of about 80-120° C., preferably about 100° C.; and
isolating the product compound of the formula (I).

In another embodiment of the invention there is a process as described in the embodiment immediately above, and wherein:
providing an aryl halide of the formula (II);
R₁ is chosen from C1-6 alkyl, phenyl or C3-6 cycloalkyl optionally substituted by C1-4 alkyl and C1-4 alkoxy each of the above may be partially or fully halogenated;
R₂ is chosen from C1-6 alkyl, C3-6 cycloalkyl optionally substituted by C1-4 alkyl, each of the above may be partially or fully halogenated and chloro.

In another embodiment of the invention there is a process as described in the embodiment immediately above, and wherein:
R₁ is C1-3 alkoxy optionally partially or fully halogenated;
R₂ is chosen from C1-6 alkyl, C3-6 cycloalkyl optionally substituted by C1-3 alkyl, each of the above may be partially or fully halogenated and chloro.

The following are representative compounds which can be made by the process described herein:

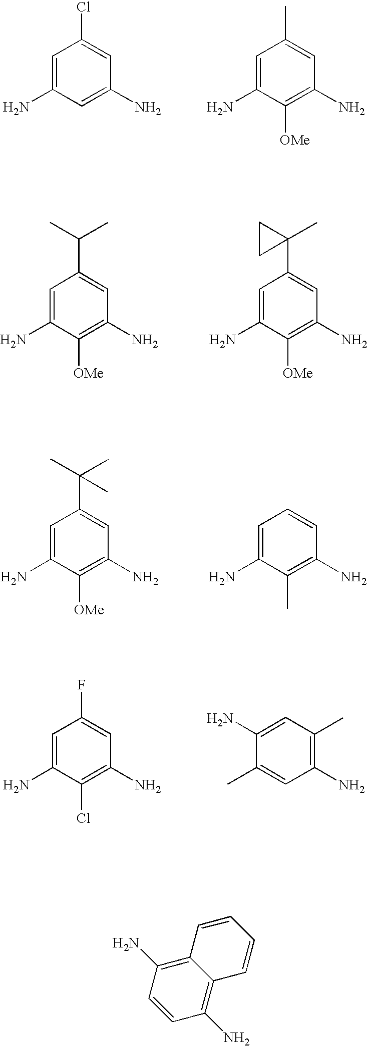

### SYNTHETIC EXAMPLES

### Example 1

### General Procedure A

LiHMDS (803 mg, 4.8 mmol, 2.4 equiv.) and 4 mL toluene were added to the aryl halide (2.0 mmol), triphenylsilylamine (1.32 g, 4.8 mmol, 2.4 equiv.), Pd₂(dba)₃ (74 mg, 0.08 mmol, 4 mol %) and 2-(dicyclohexylphosphino)biphenyl (68 mg, 0.19 mmol, 9.6 mol %). The reaction mixture was heated to 100° C. for 17 h. The mixture was cooled to 25° C. and quenched with IN HCl (5 mL). The mixture was stirred for 5 min and basified to pH 12 with 1N NaOH. The mixture was stirred for 5 min, the layers separated and the organic layer concentrated. The residue was dissolved in 10 mL EtOAc and p-toluenesulfonic acid (760 mg, 4.0 mmol, 2.0 equiv.) was added. The precipitate was filtered and partitioned between 10 mL water and 10 mL EtOAc. The aqueous layer was basified to pH 12 with 1N NaOH. The layers were separated. The organic layer was dried over Na₂SO₄ and concentrated.
2,6-Diamino-4-methylanisole

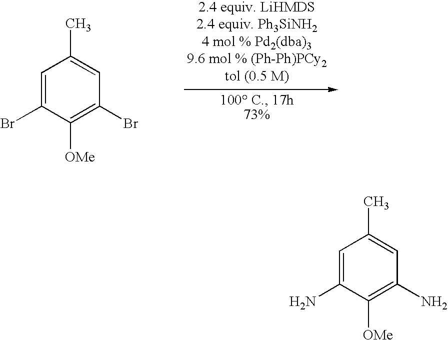

General Procedure A was followed using 2,6-dibromo-4-methylanisole (5.6 g, 20 mmol), triphenylsilylamine (13.2 g, 48 mmol, 2.4 equiv.), Pd₂(dba)₃ (740 mg, 0.8 mmol, 4 mol %), 2-(dicyclohexylphosphino)biphenyl (680 mg, 1.9 mmol, 9.6 mol %), LiHMDS (8 g, 48 mmol, 2.4 equiv.) and 40 mL toluene. The reaction mixture was heated to 100° C. for 17 h. The mixture was cooled to room temperature and quenched with IN HCl (50 mL). The mixture was stirred at room temperature for 5 min and basified to pH 12 with 1N NaOH. The mixture was stirred for 5 min, the layers separated and the organic layer was concentrated. The residue was dissolved in 100 mL EtOAc and p-toluenesulfonic acid (7.6 g, 40 mmol, 2.0 equiv.) was added. The precipitate was filtered and partitioned between 100 mL water and 100 mL EtOAc. The aqueous layer was basified to pH 12 with 1N NaOH and the layers were separated. The organic layer was dried over Na₂SO₄ and concentrated. The product was isolated as an orange oil in 70% yield (2.15 g). ¹H NMR (400 MHz, CDCl₃): δ 6.02 (s, 2 H), 3.76 (br s overlapping s, 4 H+3 H), 2.17 (s, 3 H); ¹³C NMR (100 MHz, CDCl₃): δ 139.4, 134.4, 132.5, 106.9, 59.0, 20.9; HRMS calcd for C₈H₁₃N₂O (M+H) 153.1022, found 153.1021.

[7142-138]
2,6-Diaminotoluene

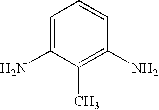

General Procedure A was followed using 1,3-dibromotoluene (500 mg, 2.0 mmol). The product was isolated as a brown solid in 86% yield (220 mg). ¹H NMR (400 MHz, CDCl₃): δ 6.84 (t, J=7.8, 1 H), 6.20 (d, J=7.8, 2 H), 3.60-3.45 (br s, 4 H), 2.05 (s, 3 H); ¹³C NMR (100 MHz, CDCl₃): δ 145.1, 126.7, 107.2, 106.6, 10.2; HRMS calcd for C₇H₁₁N₂O (M+H) 123.0916, found 123.0921.
2-Chloro-5-fluorobenzene-1,3-diamine

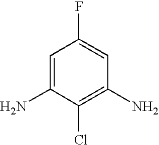

General Procedure A was followed using 1-chloro-2,6-dibromo-4-fluorobenzene (577 mg, 2.0 mmol). The product was isolated as a brown-red solid in 87% yield (280 mg). ¹H NMR (400 MHz, CDCl₃): δ 5.93 (d, J=10.1, 2 H), 4.09 (br s, 4 H); ¹³C NMR (100 MHz, CDCl₃): δ 163.6, 161.2, 144.4, 144.3, 92.6, 92.3; HRMS calcd for C₆H₇N₂FCl (M +H) 161.0276, found 161.0282.

[7142-134]
2,5-Diamino-1,4-xylene

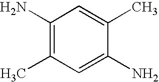

General Procedure A was followed using 2,5-dibromo-1,4-xylene (528 mg, 2.0 mmol). The product was isolated as a red oil in 66% yield (180 mg). ¹H NMR (400 MHz, CDCl₃): δ 6.40 (s, 2 H), 3.30-3.05 (br s, 4 H), 2.10 (s, 6 H); ¹³C NMR (100 MHz, CDCl₃): δ 136.6, 121.5, 117.9, 17.0; HRMS calcd for C₈H₁₃N₂ (M+H) 137.1073, found 137.1069.
1,4-Diaminonaphthalene

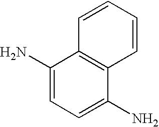

General Procedure A was followed using 1,4-dibromonaphthalene (572 mg, 2.0 mmol). The product was isolated as a yellow solid in 76% yield (240 mg). ¹H NMR (400 MHz, CDCl₃): δ 7.87 (m, 2 H), 7.49 (m, 2 H), 6.68 (s, 2 H), 3.80 (br s, 4 H); ¹³C NMR (100 MHz, CDCl₃): δ 134.8, 125.0, 121.7, 110.9; HRMS calcd for C₁₀H₁₀N₂ (M+H) 158.0843, found 158.0837.
2,6-Diamino-4-isopropylanisole

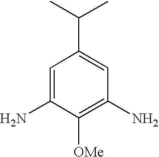

General Procedure A was followed using 2,6-dibromo-4-isopropylanisole (616 mg, 2.0 mmol). The product was isolated as an orange oil in 74% yield (266 mg). ¹H NMR (400 MHz, CDCl₃): δ 6.10 (s, 2 H), 3.84 (br s, 4 H), 3.76 (s, 3 H), 2.68 (septuplet, J=6.9, 1 H), 1.17 (d, J=6.9, 6 H); ¹³C NMR (100 MHz, CDCl₃): δ 145.9, 139.2, 133.1, 104.9, 58.6, 33.8, 23.9; HRMS calcd for C₁₀H₁₇N₂O (M+H) 181.1335, found 181.1337.
2,6-Diamino-4-tert-butylanisole

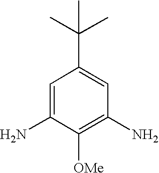

General Procedure A was followed using 2,6-dibromo-4-tert-butylanisole (644 mg, 2.0 mmol). The product was isolated as an orange oil in 69% yield (268 mg). ¹H NMR (400 MHz, CDCl₃): δ 6.23 (s, 2 H), 3.75 (s, 3 H overlapping br s, 4 H), 1.24 (s, 9 H); ¹³C NMR (100 MHz, CDCl₃): δ 148.1, 139.1, 132.7, 103.9, 58.4, 34.2, 31.3; HRMS calcd for C₁₁H₁₉N₂O (M+H) 195.1491, found 195.1500.

### Example 2

### General Procedure B

LiHMDS (12.2 g, 73.1 mmol, 2.6 equiv.) and 90 mL toluene were added to the aryl halide (28 mmol), triphenylsilylamine (20.1 g, 73.1 mmol, 2.6 equiv.), Pd₂(dba)₃ (515 mg, 0.6 mmol, 2 mol %) and 2-(dicyclohexylphosphino)biphenyl (475 mg, 1.3 mmol, 4.8 mol %). The reaction mixture was heated to 100° C. for 17 h. The mixture was cooled to 25° C., quenched with 1N HCl (30 mL) and neutralized to pH 8-9 with 3N NaOH. The mixture was stirred for 5 min, the layers separated and the organic layer was concentrated under reduced pressure. The residue was dissolved in 100 mL MTBE and p-toluenesulfonic acid (10.6 g, 60.0 mmol, 2.1 equiv.) was added. The precipitate was filtered and taken in 50 mL water and 100 mL MTBE. The aqueous layer was basified to pH 10 with 3N NaOH. The layers were separated and the organic layer was dried over Na₂SO₄ and concentrated.
2-Methoxy-5-(1-methyl-cyclopropyl)-benzene-1,3-diamine

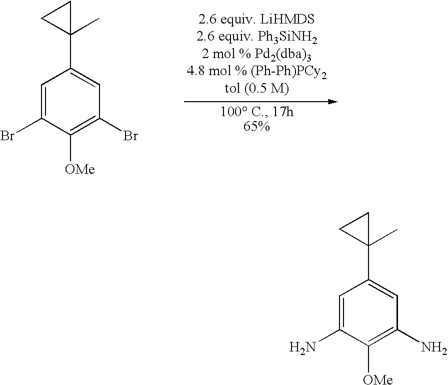

General Procedure B was followed using 1,3-dibromo-2-methoxy-5-(1-methylcyclopropyl)-benzene (9.0 g, 28.0 mmol). The product was isolated as a deep red oil in 65% yield (3.6 g) and 96% purity (by ¹H NMR assay). ¹H NMR (400 MHz, CDCl₃): δ 6.10 (s, 2 H), 4.08 (br s, 4 H), 3.68 (s, 3 H), 1.24 (s, 3 H), 0.69 (m, 2 H), 0.54 (m, 2 H); ¹³C NMR (100 MHz, CDCl₃): δ 144.1, 138.7, 133.4, 106.0, 58.8, 25.9, 19.5, 15.4; HRMS calcd for C₁₁H₁₇N₂O (M+H) 193.1335, found 193.1336.

### Example 3

### General Procedure C

To the aryl halide (2.0 mmol), Pd₂(dba)₃ (37 mg, 0.04 mmol, 2 mol %) and 2-(dicyclohexylphosphino)biphenyl (34 mg, 0.1 mmol, 4.8 mol %) were added LiHMDS (803 mg, 4.8 mmol, 2.4 equiv.) and 4 mL toluene. The reaction mixture was stirred at room temperature for 17 h. At reaction completion, the mixture was quenched with 1N HCl (5 mL) and stirred at room temperature for 5 min. Then, it was basified to pH=12 with 1N NaOH and the layers were separated. The organic layer was concentrated.
5-Chlorobenzene-1,3-diamine

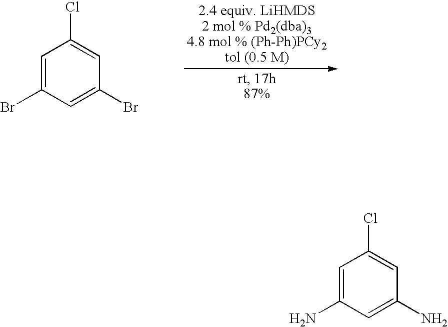

General Procedure C was followed using 5-chloro-1,3-dibromobenzene (540 mg, 2.0 mmol). The product was isolated as a brown oil in 97% yield (299 mg, 105% mass recovery and 83% purity). ¹H NMR (400 MHz, CDCl₃): δ 6.10 (s, 2 H), 5.87 (s, 1 H), 3.60 (br s, 4 H); ¹³C NMR (100 MHz, CDCl₃): δ 148.3, 135.5, 105.9, 99.7; HRMS calcd for C₆H₈N₂Cl (M+H) 143.0370, found 143.0369.

1. A process of making a compound of the formulas (I) or (III): 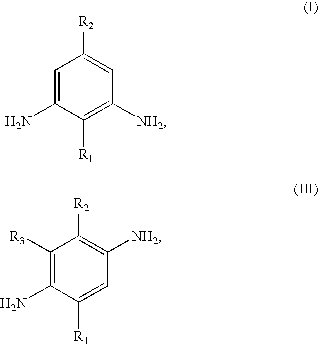
wherein
R₁ is chosen from hydrogen, C1-6 alkyl, aryl or C3-7 cycloalkyl each optionally substituted by C1-6 alkyl, C1-4 acyl, aroyl, C1-4 alkoxy, C1-6 alkoxycarbonyl each of the above may be partially or fully halogenated, carbocyclesulfonyl and —SO₂—CF₃;
R₂ is chosen from hydrogen, C1-6 alkyl, C3-7 cycloalkyl optionally substituted by C1-6 alkyl, C1-4 acyl, aroyl, C1-4 alkoxy, C1-6 alkoxycarbonyl each of the above may be partially or fully halogenated, carbocyclesulfonyl, halogen and —SO₂—CF₃;
wherein for formula II, R₃ and R₂ optionally fuse to form a benzo ring;
the process comprising in a one pot reaction:
providing an aryl halide of the formula (II) for making formula (I) or providing an aryl halide of the formula (IV) for making formula (III): 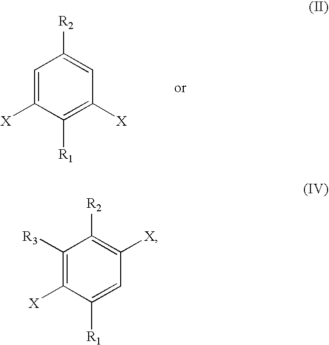
wherein R₁, R₂, R₃ are as defined above, each X is independently halogen chosen from I and Br;
adding the following in a suitable aprotic solvent:
an ammonia containing compound;
a palladium containing compound;
a phosphine containing compound;
and LiHMDS (lithium bis-trimethylsiloamide);
at a temperature of about 80-120° C., and
isolating the product compound of the formulas (I) or (III).

2. The process according to claim 1 wherein:
the process is for making formula (I);
the process comprises proving a compound of the formula (II);
R₁ is chosen from C1-6 alkyl, phenyl or C3-6 cycloalkyl optionally substituted by C1-4 alkyl and C1-4 alkoxy each of the above may be partially or fully halogenated;
R₂ is chosen from C1-6 alkyl, C3-6 cycloalkyl optionally substituted by C1-4 alkyl, each of the above may be partially or fully halogenated and chloro;
the aprotic solvent is toluene, THF or dioxane;
the ammonia containing compound is triphenylsilylamine, tri-n-hexylsilylamine, trimethylsilylamine, t-butyl carbamate or benzyl carbamate;
the palladium containing compound is Pd₂(dba)₃, Pd(dba)₂, Pd(OAc)₂ PdCl₂ or [(allyl)PdCl]₂;
the phosphine containing compound is 2-(dicyclohexylphosphino)biphenyl, triphenylphosphine, tri-t-butylphosphine, BINAP or DPPF; and
the temperature is about 100° C.

3. The process according to claim 2 wherein:
the aprotic solvent is toluene;
the ammonia containing compound is triphenylsilylamine;
the palladium containing compound is Pd₂(dba)₃; and
the phosphine containing compound is 2-(dicyclohexylphosphino)biphenyl.

4. The process according to claim 3 wherein:
R₁ is C1-3 alkoxy optionally partially or fully halogenated;
R₂ is chosen from C1-6 alkyl, C3-6 cycloalkyl optionally substituted by C1-3 alkyl, each of the above may be partially or fully halogenated and chloro.
